(19) United States
(12) Patent Application Publication
(10) Pub. No.: US 2025/0249486 A1
(43) Pub. Date: Aug. 7, 2025

(54) MEASURING APPARATUS FOR MEASURING CHEMICAL OR PHYSICAL CHARACTERISTICS OF A FLUID, AND CLEANING METHOD

(71) Applicant: Airbus Operations GmbH, HAMBURG (DE)

(72) Inventors: Tobias MEYER, HAMBURG (DE); Jan SCHILLER, HAMBURG (DE); Benjamin TEICH, HAMBURG (DE)

(21) Appl. No.: 19/045,137

(22) Filed: Feb. 4, 2025

(30) Foreign Application Priority Data
Feb. 6, 2024 (EP) 24156168.7

(51) Int. Cl. B08B 1/30; B08B 1/16; G01N 27/22; G01N 33/44

(52) U.S. Cl. CPC B08B 1/30; B08B 1/16; G01N 27/226; G01N 33/442

(57) ABSTRACT

A measuring apparatus for measuring chemical or physical characteristics of a fluid in real time, wherein a measuring window of a fluid channel can be cleaned by an internal cleaning device having an extendable, movable, and rotatable wiper element. Also a sensor carrier for the measuring apparatus and a cleaning method.

### CROSS-REFERENCES TO RELATED APPLICATIONS

This application claims the benefit of European Patent Application Number 24156168.7 filed on Feb. 6, 2024, the entire disclosure of which is incorporated herein by way of reference.

### FIELD OF THE INVENTION

The present invention refers to a measuring apparatus for measuring chemical or physical characteristics of a fluid, a sensor carrier for a measuring apparatus for measuring chemical or physical characteristics of a fluid, a cleaning device for a measuring apparatus for measuring chemical or physical characteristics of a fluid, and a method for cleaning a measuring window of a fluid channel.

### BACKGROUND OF THE INVENTION

Liquor mixtures or gas mixtures are regularly monitored regarding their chemical and/or physical characteristics. For instance, in the field of fiber composites, the mixture ratio of the resin components is checked. This can be done by feeding the resin through a fluid channel and using a sensor means. When the resin flows through the channel, contaminations/deposits can be formed on the channel wall. As a consequence, in order to avoid inaccurate measurements or readings, at least a sensor means related channel wall area has to be cleaned regularly by removing the deposits. For this purpose, the channel is opened from time to time such that it is split into two longitudinal halves and can be cleaned up. However, such cleaning is very time-consuming.

### SUMMARY OF THE INVENTION

It is an object of the present invention to provide a measuring apparatus for measuring chemical or physical characteristics of a fluid, which enables a high operating time, a sensor carrier for such measuring apparatus, a cleaning device for such measuring apparatus which enables a fast cleaning of a sensor means related fluid channel wall area, and a reliable cleaning method for cleaning a sensor means related fluid channel wall area.

The object may be achieved by a measuring apparatus with the features of one or more embodiments described herein, by a sensor carrier with the features of one or more embodiments described herein, by a cleaning device with the features of one or more embodiments described herein, and by a method with the features of one or more embodiments described herein.

According to the invention, a measuring apparatus for measuring chemical or physical characteristics of a fluid, has a sensor carrier with a fluid channel inside which is adapted to feed a fluid through the sensor carrier and a sensor means which is adapted to measure at least one chemical or physical characteristic of the fluid within the fluid channel. A measuring window through which the sensor means can communicate with the fluid inside the channel forms a wall area of the fluid channel. Further on, the measuring apparatus has a cleaning device which is adapted to remove fluid deposits from the measuring window. The cleaning device has a wiper element, which is guided at least partially inside the sensor carrier opposite to the measuring window. The wiper element can be moved relative to the measuring window in at least two directions such that the wiper element touches the measuring window.

By means of this, the measuring window can be cleaned in situ in closed environments without the need of disassembly. As a consequence, the downtime of the measuring apparatus is reduced and its operating time is enhanced. Exemplary sensor means are capacitive sensors and refractometric sensors.

In a preferred embodiment, the wiper element can be moved along its longitudinal axis crossing the fluid channel and rotationally around its longitudinal axis. By moving the wiper element transversely to the fluid channel, it can be positioned in an idle position (basic/initial position) outside the fluid flow. In addition, the force under which the wiper element acts on the measuring window can be adjusted. By rotating the wiper element around its longitudinal axis, deposits on the measuring window can be removed effectively. Preferably, the wiper element and the measuring window are rotational symmetric.

In order to reach the entire measuring window just by rotating the wiper element, a front face part of the wiper element which touches the measuring window has an extension perpendicular to the longitudinal axis (rotational axis) of the wiper element is at least equal the greatest extension of the measuring window in the cross direction. In one embodiment, the front face part is a wiper blade which is fixed to a head, for instance a disk-like head, of the wiper element. In another embodiment, the front face part is a head of the wiper itself.

By making the front face part of the wiper element of a material which is softer than the material the measuring window is made of, the measuring window is resistant against wear or abrasion. For instance, the front face part is made of a rubber or elastic material. If the head is the front face part itself, the head is made of the soft material.

In order to avoid turbulences in the fluid flow which could cause inaccurate measurements, in its idle position, the wiper element forms a wall area of the fluid channel.

Alternatively, in its idle position, the wiper element rests in a recess and is covered by a cover which forms a wall area of the fluid channel.

According to the invention, a sensor carrier for a measuring apparatus for measuring chemical or physical characteristics of a fluid according to any of the preceding claims, comprises a body, a fluid channel which is formed inside the body, a body section for receiving a measuring window forming a wall area of the fluid channel, and a body section for receiving at least partially a cleaning device which is positioned opposite to the body section for receiving the measuring window.

Such a sensor carrier enables a cleaning of the measuring window without being disassembled. Consequently, the sensor carrier could be a single part as an opening of the fluid channel is not needed for cleaning the measuring window. However, for measuring reasons of the fluid channel at a whole, the sensor carrier may have a lower half and an upper half, whose separation plane runs through the fluid channel in longitudinal direction.

According to the invention, a cleaning device is adapted to be installed in an inventive measuring apparatus and/or to be installed in an inventive sensor carrier.

According to the invention, a method for cleaning a measuring window of a fluid channel, wherein the measuring window forms a wall part of a fluid channel inside a sensor carrier, comprises the steps:
Extending a wiper element of a cleaning device from an idle position inside the sensor carrier in a working position, in which it touches the measuring window of the sensor means;
Moving the wiper element on the measuring window until fluid deposits are removed from the measuring window; and
Retracting the wiper element back in its idle position;
Such cleaning method enables a reliable and fast cleaning of the measuring window.

Preferably, cleaning parameters such as a pressure force, rotational speed, rotational direction of the wiper element, and/or duration of the cleaning process can be adjusted. By adjusting the cleaning parameters individually, the deposits can be removed optimally from the measuring window.

### BRIEF DESCRIPTION OF THE DRAWINGS

In what follows, preferred embodiments of the present invention are explained with respect to the accompanying drawings. As is to be understood, the various elements and components are depicted as examples only, may be facultative and/or combined in a manner different than that depicted. Reference signs for related elements are used comprehensively and not necessarily defined again for each figure. Shown is schematically in:

FIG. 1 a longitudinal section of an inventive measuring apparatus before cleaning its measuring window;

FIG. 2 a longitudinal section of an inventive measuring apparatus during cleaning the measuring window;

FIG. 3 a longitudinal section of an inventive measuring apparatus after cleaning the measuring window;

FIG. 4 a longitudinal section of an alternative inventive measuring apparatus during cleaning the measuring window;

FIG. 5 an inventive cleaning device with its wiper element in a retracted state;

FIG. 6 the cleaning device with its wiper element in an extended state; and

FIG. 7 a front view of an exemplary head of the wiper element.

### DETAILED DESCRIPTION OF THE PREFERRED EMBODIMENTS

In FIG. 1, a first embodiment of a measuring apparatus 1 according to the invention is shown. The measuring apparatus 1 can be used for measuring at least one chemical or physical characteristics of a fluid (liquor of gas) in real time. An exemplary fluid is a resin used for fiber composites, such as CFRP or GFRP. The characteristic which should be measured is the mixture ratio of the resin. The measuring apparatus can be an integral part of a forming/molding tooling or separate part of such tooling.

The measuring apparatus 1 has a sensor carrier 2 in which a fluid channel 4 is provided. The fluid channel 4 extends through the sensor carrier 2 and has preferably a rectangular-like cross section, which is delimited by its channel wall 6. However, the fluid channel 4 can also have a different cross section such as a circular cross section. It is also possible, that the cross section of the fluid channel varies. For instance, along a measuring path, the cross section can be rectangular and in a channel area before and/or after the measuring path, the fluid channel 4 can has a circular cross section.

The fluid channel 4 is provided to feed a fluid to be monitored through the sensor carrier 2. For this purpose, the sensor carrier 2 can be integrated into a not shown fluid system, feeding the fluid to the fluid channel 4 and discharging the fluid from the fluid channel afterwards.

In order to measure the at least one fluid characteristic, the measuring apparatus 1 comprises a sensor means 8. Further on, the measuring apparatus 1 comprises a cleaning device 10. Both, the sensor means 8 and the cleaning device 10 are positioned at least partially in a recess 12, 14 of the sensor carrier 2.

In this example, the sensor means 8 is a capacitive sensor. A front measuring surface of the sensor builds a part of the fluid channel wall 6 and acts as a measuring window 16 of the fluid channel 4. For instance, the measuring surface, and such the measuring window 16, can be a metallic, flat surface.

The cleaning device 10 is positioned in its carrier recess 14 opposite to the sensor means 8. It is used to remove contaminations/deposits 18 of the fluid from the measuring window 16, preferably before a new measurement series starts. The cleaning device 10 has a wiper element 20 which is guided inside a not shown bearing or bushing arrangement and whose movable and rotatable shaft 22 is sealed against fluid leakage.

The wiper element 20 can be moved in two directions. The first direction 24 corresponds to its longitudinal axis x and the second direction corresponds to a rotation 26 around the longitudinal axis x. The longitudinal axis x of the wiper element 20 extends orthogonally to the measuring window 16 and orthogonally to a central axis c of the fluid channel 4. In order to enable a rotation of the wiper element 20 on the measuring window 16, both a head 28 of the wiper element 20 touching the measuring window 16 for cleaning reasons and the measuring window 16 are rotationally symmetrically.

If not in use, in particular during a measurement, the wiper element 20 rests in an idle position. In its basis position the wiper element 20 is positioned outside of a fluid flow. In this example, the head 28 of the wiper element 20 forms an area of the channel wall 6. As shown in FIG. 1, the head 28 of the wiper element 20 is also contaminated.

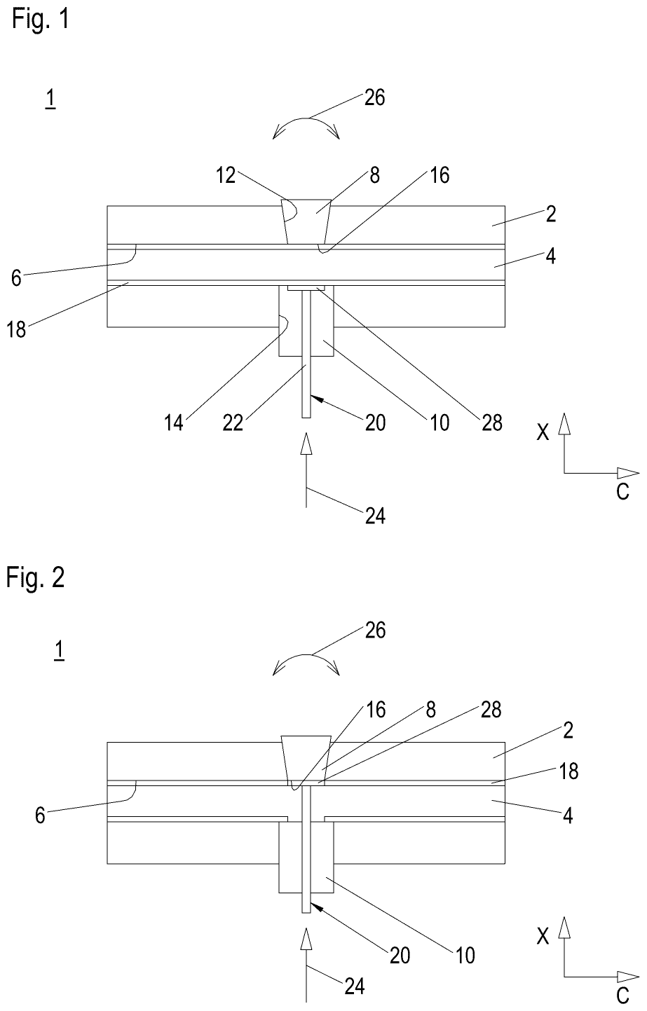

Before a new measurement series starts, all contamination/deposits 18 will be removed from the measuring window 16. For this purpose, as shown in FIG. 2, the wiper element 20 extends from its idle position and is moved along its longitudinal axis x across the fluid channel 4 in its working position. In its working position, its head 28 touches the measuring window 16 and rotates around its longitudinal axis x. In order to achieve an optimal cleaning result, a pressure force, rotational speed, rotational direction and/or duration of the cleaning process can be adjusted as a function of the kind of contamination/deposits 18. As a consequence, the contamination 18 on the measuring window 16 and on the head 28 are removed (wiped off).

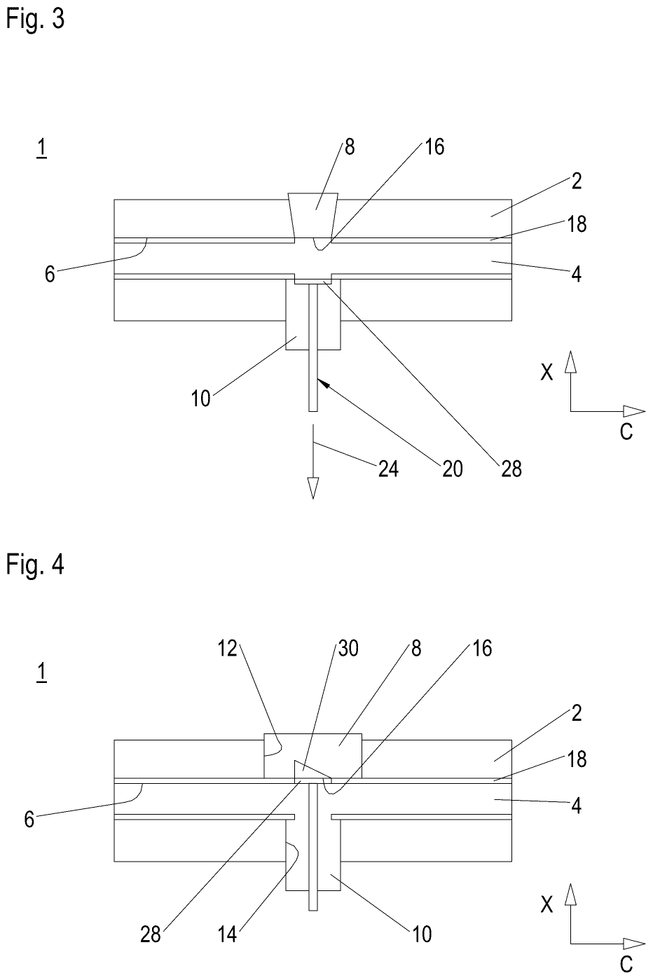

After the cleaning of the measuring window 16 is terminated, the rotation is stopped and the wiper element 20 is retracted back into its idle position, as shown in FIG. 3. Now, the measuring apparatus is prepared for a new measurement.

In FIG. 4, an alternative measuring apparatus 1 is shown in a working position of a wiper element 20. Contrary to the measuring apparatus 1 described in FIGS. 1, 2 and 3, the alternative measuring apparatus 1 is equipped with a sensor means 8 based on refractometry (refractometric sensor). For example, a prism 30 is provided via whom light waves are emitted into a fluid flow and received back. Here, a boundary layer of the prism 30 to the fluid channel 4 forms the measuring window 16 of the fluid channel 4.

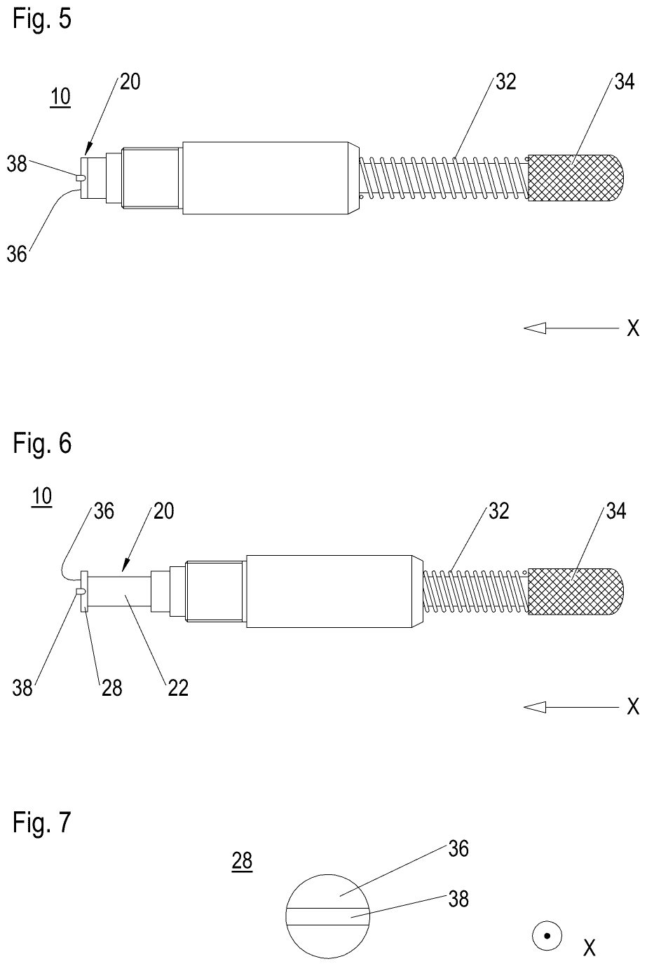

In FIGS. 5 and 6, an exemplary cleaning device 10 is shown in its idle position (FIG. 5) and its working position (FIG. 6). In the idle position, the wiper element 20 is retracted. In the working position, the wiper element 20 is extended. In order to facilitate the retraction movement, on a backside of the cleaning element 10, a spring 32 is provided by means of which the wiper element 20 is preloaded in its idle position. In addition, a rear handle 34 can be provided in order to operate the cleaning device 10 manually. In a preferred mode, the cleaning device 10 is operated fully automatically.

As also illustrated in more detail in FIGS. 5, 6 and 7, the head 28 of the wiper element 20 has a disc-like shape and comprises at its front face 36 a wiper blade 38. The wiper blade 38 has a longitudinal shape and extends through a disc center radially on the front face 36 of the head 28. In particular, the wiper blade 38 has an extension transversely to the longitudinal axis a (rotational axis) of the wiper element 20 that is at least equal to the greatest extension of the measuring window 16 in the cross direction. The wiper blade 38 is held in a groove of the head 28 and made of a material which is softer than the material of the measuring window 16. For instance, the wiper blade 38 is made of an elastic or rubber like material whereas the measuring window is made of glass. Thereby, it is important that during a cleaning process the wiper blade 38 does not cause any streaks, smudges or similar effects on the measuring window 16, which could have a negative impact on the measurement result. If the wiper blade 38 is worn-out, it will be replaced by a new one. Then, the cleaning mean 10 will be removed from the sensor carrier 2 backwards and inserted into the recess after the wiper blade 38 has been renewed. When removing and replacing the used cleaning device 10, an assembled state of the sensor carrier 2 can be maintained. Both, the cleaning device 10 and the sensor means 8 can be hold in place via a screw connection, a snap- and click connection and the like.

As the wiper blade 38 extends over the front face 36 of the head 28 in longitudinal direction x of the wiper element 20, the head 28 always touches the measuring window 16 via the wiper blade 38.

The systems and devices described herein may include a controller or a computing device comprising a processing unit and a memory which has stored therein computer-executable instructions for implementing the processes described herein. The processing unit may comprise any suitable devices configured to cause a series of steps to be performed so as to implement the method such that instructions, when executed by the computing device or other programmable apparatus, may cause the functions/acts/steps specified in the methods described herein to be executed. The processing unit may comprise, for example, any type of general-purpose microprocessor or microcontroller, a digital signal processing (DSP) processor, a central processing unit (CPU), an integrated circuit, a field programmable gate array (FPGA), a reconfigurable processor, other suitably programmed or programmable logic circuits, or any combination thereof.

The memory may be any suitable known or other machine-readable storage medium. The memory may comprise non-transitory computer readable storage medium such as, for example, but not limited to, an electronic, magnetic, optical, electromagnetic, infrared, or semiconductor system, apparatus, or device, or any suitable combination of the foregoing. The memory may include a suitable combination of any type of computer memory that is located either internally or externally to the device such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like. The memory may comprise any storage means (e.g., devices) suitable for retrievably storing the computer-executable instructions executable by processing unit.

The methods and systems described herein may be implemented in a high-level procedural or object-oriented programming or scripting language, or a combination thereof, to communicate with or assist in the operation of the controller or computing device. Alternatively, the methods and systems described herein may be implemented in assembly or machine language. The language may be a compiled or interpreted language. Program code for implementing the methods and systems described herein may be stored on the storage media or the device, for example a ROM, a magnetic disk, an optical disc, a flash drive, or any other suitable storage media or device. The program code may be readable by a general or special-purpose programmable computer for configuring and operating the computer when the storage media or device is read by the computer to perform the procedures described herein.

Computer-executable instructions may be in many forms, including modules, executed by one or more computers or other devices. Generally, modules include routines, programs, objects, components, data structures, etc., that perform particular tasks or implement particular abstract data types. Typically, the functionality of the modules may be combined or distributed as desired in various embodiments.

It will be appreciated that the systems and devices and components thereof may utilize communication through any of various network protocols such as TCP/IP, Ethernet, FTP, HTTP and the like, and/or through various wireless communication technologies such as GSM, CDMA, Wi-Fi, and WiMAX, is and the various computing devices described herein may be configured to communicate using any of these network protocols or technologies.

Disclosed is a measuring apparatus for measuring chemical or physical characteristics of a fluid in real time, wherein a measuring window of a fluid channel can be cleaned by an internal cleaning device having an extendable and rotatable wiper element, thus avoiding to open a fluid channel longitudinally for cleaning purposes of the measuring window, a sensor carrier for the measuring apparatus and a cleaning method.

While at least one exemplary embodiment of the present invention(s) is disclosed herein, it should be understood that modifications, substitutions and alternatives may be apparent to one of ordinary skill in the art and can be made without departing from the scope of this disclosure. This disclosure is intended to cover any adaptations or variations of the exemplary embodiment(s). In addition, in this disclosure, the terms “comprise” or “comprising” do not exclude other elements or steps, the terms “a” or “one” do not exclude a plural number, and the term “or” means either or both. Furthermore, characteristics or steps which have been described may also be used in combination with other characteristics or steps and in any order unless the disclosure or context suggests otherwise. This disclosure hereby incorporates by reference the complete disclosure of any patent or application from which it claims benefit or priority.

### REFERENCE LIST

1 measuring apparatus
2 sensor carrier
4 fluid channel
6 channel wall
8 sensor means
10 cleaning device
12 recess for sensor means/body section
14 recess for cleaning device/body section
16 measuring window
18 contamination/deposits
20 wiper element
22 shaft
24 first direction
26 second direction
28 head of the wiper element
30 prism
32 spring
34 handle
36 front face
38 wiper blade/front face part
X longitudinal axis of the fluid channel
c central axis (rotational axis) of the wiper element

1. A measuring apparatus for measuring chemical or physical characteristics of a fluid, the measuring apparatus comprising:
a sensor carrier having a fluid channel configured to feed a fluid through the sensor carrier;
a measuring window forming a wall area of the fluid channel;
a sensor means configured to measure at least one chemical or physical characteristic of the fluid within the fluid channel through the measuring window,
and
a cleaning device configured to remove fluid deposits from the measuring window,
wherein the cleaning device has a wiper element, which is guided, at least partially, inside the sensor carrier opposite to the measuring window, and
wherein the wiper element is configured to move relative to the measuring window in at least two directions such that the wiper element touches the measuring window.

2. The measuring apparatus according to claim 1, wherein the wiper element is configured to move along a longitudinal axis of the wiper element crossing the fluid channel and to move rotationally around the longitudinal axis.

3. The measuring apparatus according to claim 1, wherein a front face part of the wiper element touching the measuring window has an extension cross to 1 longitudinal axis of the wiper element that is at least equal to a greatest extension of the measuring window in a transversal direction of the measuring window.

4. The measuring apparatus according to claim 1, wherein the wiper element has a front face part made of a material which is softer than a material that the measuring window is made of.

5. The measuring apparatus according to claim 1, wherein a its idle position, the wiper element forms a wall area of the fluid channel.

6. The measuring apparatus according to claim 1, wherein in a idle position, the wiper element is covered by a cover which forms a wall area of the fluid channel.

7. A sensor carrier for the measuring apparatus according to claim 1, the sensor carrier comprising:
a body,
the fluid channel formed inside the body;
a body section for receiving the measuring window forming the wall area of the fluid channel; and
an opposite body section for receiving, at least partially, the cleaning device.

8. A method for cleaning a measuring window of a fluid channel, wherein the measuring window forms a wall part of a fluid channel inside a sensor carrier, the method comprising the steps:
extending a wiper element of a cleaning device from an idle position inside the sensor carrier in a working position, in which the wiper element touches the measuring window of a sensor means in the senor carrier;
moving the wiper element on the measuring window until fluid deposits are removed from the measuring window; and
retracting the wiper element back in the idle position.

9. The method according to claim 8, wherein a pressure force, a rotational speed, a rotational direction, a duration of a cleaning process, or any combination thereof are adjusted.
